(19) United States
(12) Patent Application Publication
(10) Pub. No.: US 2024/0189470 A1
(43) Pub. Date: Jun. 13, 2024

(54) BODY SUPPORT APPARATUS

(71) Applicant: Beyond CenterLine Solutions, Colorado Springs, CO (US)

(72) Inventor: Richard Michael Bond, Colorado Springs, CO (US)

(73) Assignee: Beyond CenterLine Solutions, Colorado Springs, CO (US)

(21) Appl. No.: 18/064,017

(22) Filed: Dec. 9, 2022

(51) Int. Cl. A61L 2/24; A61L 2/10; A61L 2/238; B08B 1/00; B08B 13/00

(52) U.S. Cl. CPC A61L 2/24; A61L 2/10; A61L 2/238; B08B 1/006; B08B 13/00; A61L 2202/11; A61L 2202/14; A61L 2202/17

(57) ABSTRACT

A body support apparatus is disclosed. The body support apparatus may include a mounting surface configured to be disposed on a structure, such as a wall. The body support apparatus may further include a touch surface opposite to the mounting surface. The touch surface may include a textured hand support surface configured to provide support to a user while leaning against the structure. The touch surface may further include a handprint. The handprint may include a luminescent surface that may glow in dark ambient environment. The body support apparatus may further include a cleansing mechanism configured to clean the touch surface. The cleansing mechanism may include a disinfectant light mechanism configured to disinfect the touch surface using ultraviolet light.

### TECHNICAL FIELD

The present disclosure relates to a body support apparatus, and more specifically to a body support apparatus mountable on a wall that enables a user to lean against the wall without disfiguring the wall.

### BACKGROUND

People may lean against a wall or a support surface for one's convenience, physical condition, body balancing, or due to habit. For example, a person may lean against a wall while removing/putting footwear, urinating, adjusting garments, etc. People generally perform such activities using one hand and lean against the wall using the other hand.

Over time, wall surface where people rest their hands or other body parts may become dirty or marred from repeated touches. For example, a wall surface over or adjacent to a commode or urinal may be repeatedly touched by users, and may thus become dirty. Further, such surfaces may not be conveniently cleanable, and it may not be possible to clean such surfaces regularly. Unclean wall surfaces may result in hygiene issues, which may cause inconvenience to users.

Thus, there is a need for an apparatus that the users may use to lean against the wall, which may prevent wall disfigurement.

It is with respect to these and other considerations that the disclosure made herein is presented.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIG. 1 depicts an example environment in which techniques and structures for providing the systems and methods disclosed herein may be implemented.

FIG. 2 depicts an example body support apparatus in accordance with the present disclosure.

FIG. 3 depicts another example body support apparatus in accordance with the present disclosure.

FIG. 4 depicts a yet another example body support apparatus in accordance with the present disclosure.

### DETAILED DESCRIPTION

### Overview

The present disclosure is directed towards a body support apparatus that may be mounted on a wall (or any other structure). A user may use the body support apparatus to lean against the wall to perform activities such as removing or putting footwear, adjusting garments worn by the user, urination, and/or the like. The wall may be located in any location including, but not limited to, a washroom, a hallway, home entry areas, etc., where the user may need the wall support to perform the activities mentioned above. The apparatus may be a supporting pad having a textured exterior surface that may prevent slipping of user's hand when the user rests the hand on the textured exterior surface. The apparatus may be shaped as a human hand or may include a handprint to provide a visual indication to the user to rest the hand on the apparatus.

In some aspects, the apparatus may include a luminescent surface that may glow in dark ambient environment. In further aspects, the handprint may include the luminescent surface that may glow in the dark.

The apparatus may further include a self-cleaning mechanism that may disinfect the textured exterior surface using ultraviolet light (or similar mechanism). In some aspects, the self-cleaning mechanism may emit the ultraviolet light at a predetermined frequency. In other aspects, the self-cleaning mechanism may emit the ultraviolet light responsive to a trigger event (e.g., when the user is in proximity to the apparatus or is using/has used the apparatus).

In additional aspects, the apparatus may include removable films or replaceable media disposed over the textured exterior surface. The user may remove the removable film after (or before) using the apparatus. In some aspects, the apparatus may automatically replace a used film with a new and fresh film, when or after the user uses the apparatus.

In further aspects, the apparatus may include an integrated or removable box including cleansing wipes. The user may clean the textured exterior surface by using a cleansing wipe after (or before) using the apparatus.

The present disclosure discloses a body support apparatus that enables the user to lean against the wall without disfiguring the wall. The apparatus prevents hand slippage, and thus the user may balance user's body while leaning. The apparatus may be easily mounted on the wall via adhesives or fasteners. Further, the apparatus facilitates maintenance of hygiene by exterminating or mitigating bacterial and viral content.

These and other advantages of the present disclosure are provided in detail herein.

### Illustrative Embodiments

The disclosure will be described more fully hereinafter with reference to the accompanying drawings, in which example embodiments of the disclosure are shown, and not intended to be limiting.

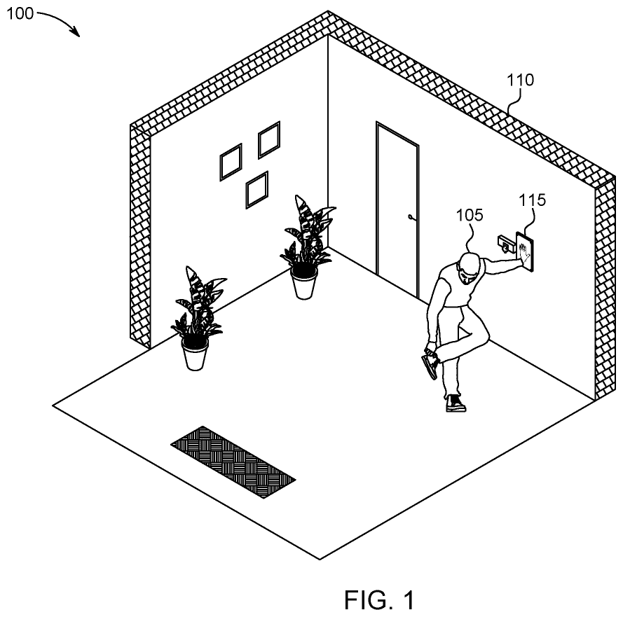

FIG. 1 depicts an example environment 100 in which techniques and structures for providing the systems and methods disclosed herein may be implemented. The environment 100 may include a user 105 who may need support of a structure (such as a wall 110) to perform one or more activities. For example, the user 105 may need support in removing or putting footwear, in adjusting garments worn by the user 105, during urination, and/or the like. The user 105 may lean against the wall 110 by using one hand, and may use the other hand for performing the activities mentioned above.

The user 105 may use a body support apparatus 115 to utilize wall 110 support (e.g., lean against the wall 110) to balance user 105 body. The body support apparatus 115 may be a supporting pad and may include a textured or roughened exterior surface, so that the user 105 may maintain grip while taking the wall 110 support. Stated another way, the body support apparatus 115 may prevent user's hand from sliding when the user 105 uses the body support apparatus 115 to lean against the wall 110.

In some aspects, the body support apparatus 115 may be mounted on the wall 110 (or any other structure like door, window, furniture, etc.) using adhesives. Specifically, the body support apparatus 115 may include the adhesives on one surface (e.g., a body support apparatus 115 inner surface that may contact the wall 110). In other aspects, the body support apparatus 115 may be mounted on the wall 110 by using fastening means such as steel socket set screws, aluminum hex head cap screws, bolts, nails, and/or the like.

The body support apparatus 115 may be mountable in any location such as a washroom, a hallway and entry areas, where the user 105 may need the wall 110 support to perform the activities mentioned above or where the user 105 may be in a habit of placing user's hand(s) on the wall 110. A person ordinarily skilled in the art may appreciate that the wall 110 may be located in any location described above.

The body support apparatus 115 may be of any size, shape, and color. For example, the body support apparatus 115 may be shaped as a human hand, so that the body support apparatus 115 may provide a visual indication to the user 105 to rest the user's hand on the body support apparatus 115. In other aspects, the body support apparatus 115 may be square or rectangular shaped with a handprint on a body support apparatus 115 outer/exterior surface (where the user 105 may rest the user's hand). In an exemplary aspect, a body support apparatus 115 length may be between 5-9 inches (or more) and a body support apparatus 115 width may be 3-6 inches (or more), so that the user 105 may conveniently rest the user hand on the body support apparatus 115. The handprint may cover between 50%-90% of the body support apparatus 115. In other aspects, the body support apparatus 115 may be circular or oval shaped.

According to other aspects, the body support apparatus 115 may be further shaped as another body part such as, for example, the buttox. As explained further below, a user may use the body support apparatus 115 to support their balance by placing their buttox against the apparatus while dressing, undressing, or performing other tasks requiring balance or support.

The body support apparatus 115 may be made of any material. For example, the body support apparatus 115 may be made of plastic, rubber or any other similar material. In some aspects, the body support apparatus 115 inner surface and the body support apparatus 115 outer surface may be made of different materials. For example, the body support apparatus 115 inner surface may be made of rubber and the body support apparatus 115 outer surface may be made of plastic. In other aspects, the body support apparatus 115 inner surface and the body support apparatus 115 outer surface may be made of same material.

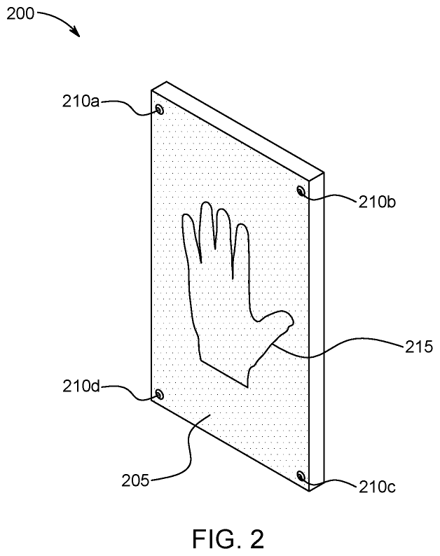

In further aspects, the body support apparatus 115 may include neon elements that may enable the body support apparatus 115 to glow in dark ambient environment. Stated another way, the body support apparatus 115 may include a luminescent surface that may glow in the dark. In some aspects, the body support apparatus 115 outer surface (that may include the handprint, as described above) may include the luminescent surface. Thus, even when the user 105 is entering or exiting a dark room, the user 105 may easily locate the body support apparatus 115 to take the support. The body support apparatus 115 may be washable and may be easily dried. The details of the body support apparatus 115 may be understood in conjunction with FIGS. 2-4.

FIG. 2 depicts an example body support apparatus 200 in accordance with the present disclosure. The body support apparatus 200 may be same as the body support apparatus 115. The body support apparatus 200 may include a mounting surface (not shown) and an outer/exterior top/exterior surface 205 opposite to the mounting surface. The mounting surface may face towards the wall 110 (or any other structure) and may be used to mount the body support apparatus 200 on the wall 110 by using adhesives and/or fastening means. The exterior surface 205 may be a hand touch surface on which the user 105 may rest/place the user hand. Hereinafter, the exterior surface 205 is referred to as the hand touch surface 205.

The hand touch surface 205 may include a textured or roughened hand support surface configured to provide support to the user hand, so that the user 105 may balance user's body while leaning against the wall 110. The textured or roughened hand support surface may ensure that the user hand does not slip when the user 105 takes support of the body support apparatus 200.

In some aspects, the hand touch surface 205 may be hand shaped. In other aspects, the hand touch surface 205 may be square or rectangular shaped having a handprint 215, as shown in FIG. 2. In some aspects, handprint 215 dimensions may be equivalent to dimensions of a standard human hand. In further aspects, the square or rectangular hand touch surface 205 may have a hand shaped cavity in which the handprint 215 may be formed. The hand shaped cavity may provide additional comfort and convenience to the user 105, when the user 105 takes apparatus 200 support. In further aspects, the hand touch surface 205 may be of any other shape such as oval, circular and/or the like.

Mounting surface shape may be same as or different from hand touch surface 205 shape. For example, the mounting surface may be square or rectangular shaped. In some aspects, the mounting surface may be of any other shape including, but not limited to, oval, circular, hand shaped and/or the like.

In some aspects, the mounting surface may be permanently or removably affixed to the wall 110 via an adhesive or a plurality of fasteners 210a, 210b, 210c, and 210d (collectively referred as fasteners 210). The fasteners 210 may be, for example, screws, nails, bolts and/or the like. A person ordinarily skilled in the art may appreciate that although four fasteners are shown in FIG. 2, the body support apparatus 200 may be mounted on the wall 110 by using more or less count of fasteners.

The mounting surface may be made of rubber or any other material (such as plastic). In some aspects, the mounting surface and the hand touch surface 205 may be made of different materials. In other aspects, the mounting surface and the hand touch surface 205 may be made of same material.

The hand touch surface 205 may be a luminescent surface that may glow in dark ambient environment, as described above in conjunction with FIG. 1. In some aspects, only the handprint 215 may glow/illuminate in the dark. In this case, the handprint 215 may be made of material including neon elements.

In further aspects, the body support apparatus 200 may have include light emitting devices (LEDs, not shown) that may illuminate the hand touch surface 205 or the handprint 215. The LEDs may be disposed along body support apparatus 200/hand touch surface 205 edges, or along a handprint 215 periphery. An apparatus battery or an external power source may power the LEDs. In an exemplary aspect, the LEDs may activate automatically when the user 105 is in proximity to the hand touch surface 205. In this case, the body support apparatus 200 may include one or more proximity sensors and a processor (not shown). The proximity sensors may detect user 105 presence in proximity to the body support apparatus 200, and may send a user proximity signal to the processor responsive to detecting the user 105 presence. The processor may cause the LEDs to activate when the processor receives the user proximity signal from the proximity sensors.

The hand touch surface 205 may additionally include an antibacterial or antiviral coating that may be configured to exterminate or mitigate bacterial and viral content that may be disposed on the hand touch surface 205. The antibacterial or antiviral coating may be a copper impregnated coating. A person ordinarily skilled in the art may appreciate that the body support apparatus 200 may maintain hygiene for apparatus 200 users by using the antibacterial or antiviral coating. The antibacterial or antiviral coating may be particularly beneficial in maintaining apparatus 200 hygiene when the body support apparatus 200 is mounted in a public place, e.g., a public urinal, where a high count of users may use the body support apparatus 200.

In further aspects, the body support apparatus 200 may include a disinfectant light mechanism that may be configured to disinfect the hand touch surface 205. In particular, the disinfectant light mechanism may use ultra-violet (UV) light to disinfect the hand touch surface 205. In some aspects, the disinfectant light mechanism may emit the UV light at a predetermined frequency, e.g., every 5 or 10 minutes. In other aspects, the disinfectant light mechanism may emit the UV light responsive to receiving a trigger event. For example, the body support apparatus 200 may include one or more sensors such as pressure sensors, proximity sensors, and/or the like, and the processor (as described above). The processor may cause the disinfectant light mechanism to emit the UV light automatically based on inputs received from the pressure sensors and/or the proximity sensors. For example, the processor may cause the disinfectant light mechanism to emit the UV light when the pressure sensors detect that the user 105 is using (or has used) the body support apparatus 200. Additionally or alternatively, the processor may cause the disinfectant light mechanism to emit the UV light when the proximity sensors detect user 105 presence in proximity to the body support apparatus 200.

In additional aspects, the body support apparatus 200 may include one or more in-built speakers. The speakers may output a preset or a customized audio message when the user 105 uses the body support apparatus 200 or when the user 105 is in apparatus 200 proximity. The apparatus battery or the external power source may power the speakers. In this case, the body support apparatus 200 may include an apparatus transceiver and an apparatus memory (not shown). In an exemplary aspect, an apparatus operator may transmit a customized message by using a user device (e.g., a mobile phone, tablet, laptop, etc.) to the apparatus transceiver or store a preset message in the apparatus memory (not shown). When the user 105 uses the body support apparatus 200 or when the user 105 is in the apparatus 200 proximity (as detected by the pressure or proximity sensors), the processor may cause the speakers to output the customized or the preset message. For example, when the body support apparatus 200 is mounted in a public urinal (e.g., in a mall), the customized message may be “Wet floor, walk cautiously”. As another example, the preset message may be an advertisement message.

According to another embodiment, a user may find it convenient to lean against a wall or other surface while undressing or dressing. For example, while preparing to bath or after bathing, the user may be accustomed to lean against a surface of the bathroom or dressing room for additional balance while dressing or undressing. While leaning for balance support, the user may position their weight such that their buttox is in contact with the wall or other surface.

Accordingly, the body support apparatus 115 may instead be configured as a backside support apparatus having an appropriate image or indicator in place of a hand print (not shown in FIG. 1 or FIG. 2). Accordingly, a user can lean against the apparatus 200 while supporting their balance using their buttox instead of a hand. In this example embodiment, the position for wall mounting the apparatus 115 may be lower and include any one or more features according to the present disclosure.

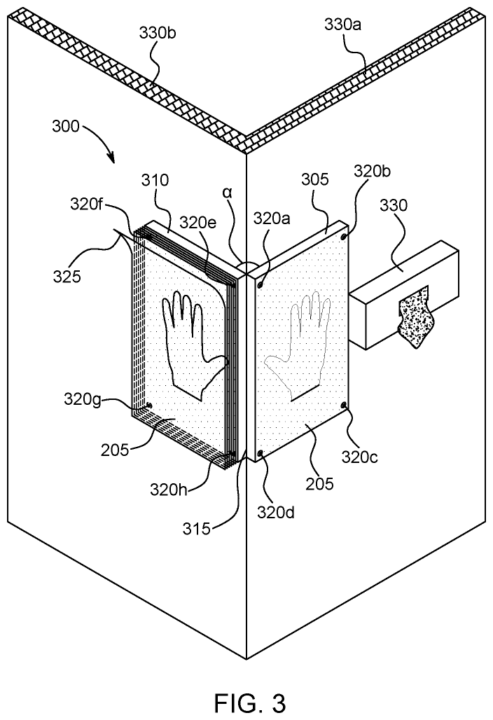

FIG. 3 depicts another example body support apparatus 300 in accordance with the present disclosure. The body support apparatus 300 may be similar to the body support apparatus 115 or 200.

In some aspects, the body support apparatus 300 may be a corner unit that may be mounted on a wall corner portion (or any other structure corner portion). The body support apparatus 300 may include a first support pad 305 and a second supporting pad 310. Each supporting pad 305, 310 may be same as the body support apparatus 115 or 200. The first and second supporting pads 305, 310 may attached with each other via a hinge at an edge 315. In some aspects, the first and second supporting pads 305, 310 may be attached in an “L” shape, such that the first and second supporting pads 305 and 310 may be mounted on adjacent walls 330a and 330b respectively. The first and second supporting pads 305, 310 may be configured to rotate relative to each other via the hinge/edge 315, such that the body support apparatus 300 may be mounted to the wall corner portion at different angles “α”. For example, the body support apparatus 300 may be mounted to the wall corner portion having a wall corner angle α of 45, 60 or 90 degrees.

In some aspects, the first and second supporting pads 305, 310 may be mounted on the adjacent walls 330a, 330b using adhesives. In other aspects, the first and second supporting pads 305, 310 may be mounted on the adjacent walls 330a, 330b using a plurality of fasteners 320a, 320b, 320c, 320d, 320e, 320f, 320g, and 320h (collectively referred as fasteners 320). A person ordinarily skilled in the art may appreciate that there may be less or more fasteners 320 than the eight fasteners shown in FIG. 3.

As described above, each supporting pad 305, 310 may include a mounting surface and an exterior surface (e.g., the hand touch surface 205 described in conjunction with FIG. 2). Each hand touch surface 205 (associated with the first and second supporting pads 305, 310) may be rectangular shaped with handprints, as shown in FIG. 3. In other aspects, the hand touch surface 205 may be hand shaped. In some aspects, the user 105 may use the first supporting pad 305 (e.g., by resting user left hand) or the second supporting pad 310 (e.g., by resting user right hand) to take support of the respective adjacent walls 330a, 330b.

The body support apparatus 300 may additionally include one or more cleaning mechanisms that may be used to keep the body support apparatus 300 clean. For example, the second supporting pad 310 may include a plurality of removable films (or replaceable media) 325 that may be disposed on the hand touch surface 205. The plurality of removable films 325 may be removably “hanged” on the second supporting pad 310 (e.g., on the hand touch surface 205). The removable films 325 may be thin films that may be made of any material such as paper, cloth, plastic and/or the like. The plurality of removable films 325 may be disposed one over the other.

The user 105 may use rest the user hand on a topmost removable film of the plurality of removable films 325 when the user 105 takes support of the second supporting pad 310. The user 105 may remove the topmost removable film when the user 105 removes the hand from the second supporting pad 310, so that a next user using the body support apparatus 300 may rest user hand on a clean and fresh removable film.

A person ordinarily skilled in the art may appreciate that although FIG. 3 depicts that the plurality of removable films 325 may be disposed on the second supporting pad 310, the plurality of removable films 325 may be disposed on the first supporting pad 305 as well. In further aspects, the plurality of removable films 325 may be disposed on only the first supporting pad 305.

In some aspects, the body support apparatus 300 may use more than one cleaning mechanism. In an exemplary aspect, the first supporting pad 305 and the second supporting pad 310 may have different cleaning mechanisms. For example, the first supporting pad 305 may have a disinfectant light mechanism (described in conjunction with FIG. 2) and the second supporting pad 310 may have the plurality of removable films 325. In other aspects, both the first supporting pad 305 and the second supporting pad 310 may have same cleaning mechanisms. For example, both the first and second supporting pads 305, 310 may have disinfectant light mechanism, or both the first and second supporting pads 305, 310 may have the plurality of removable films 325.

In further aspects, the body support apparatus 300 may include a dispenser 330 that may include cleansing wipes to clean the hand touch surface 205. The dispenser 330 may be disposed adjacent to the first supporting pad 305 and/or second supporting pad 310 (e.g., adjacent to the hand touch surface 205). In some aspects, the dispenser 330 may be located below the first supporting pad 305 and/or the second supporting pad 310. In other aspects, the dispenser 330 may be located adjacent to side edges of the first supporting pad 305 and/or the second supporting pad 310. The dispenser 330 may be integrated to the body support apparatus 300. In some aspects, the dispenser 330 may be removably attached to the body support apparatus 300.

The dispenser 330 may be an open box or a closed box with an opening to take out the cleansing wipes one by one. In some aspects, the dispenser 330 may have a lid (not shown) that may cover the open portion or the opening. The lid may be hinged at one end to enable dispenser 330 closing and opening. In further aspects, the cleansing wipes may be wet wipes that may be configured to exterminate or mitigate bacterial and viral content.

The user 105 may use a cleansing wipe to clean the hand touch surface 205 (associated with the first supporting pad 305 and/or the second supporting pad 310) when or after the user 105 uses the first supporting pad 305 and/or the second supporting pad 310. In some aspects, the user 105 may clean the hand touch surface 205 before using the hand touch surface 205. In further aspects, the body support apparatus 300 may include an integrated or removable bin (not shown) in which the user 105 may keep (or “throw”) the used cleaning wipes.

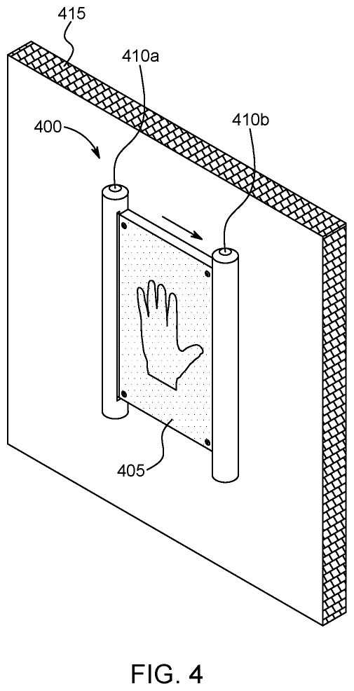

FIG. 4 depicts yet another example body support apparatus 400 in accordance with the present disclosure. The body support apparatus 400 may include a supporting pad 405 that may be same as the first supporting pad 305 or the second supporting pad 310. The supporting pad 405 may be mounted on a wall 415.

The body support apparatus 400 may include a film dispensing mechanism that may be configured to automatically supply removable films over supporting pad 405 exterior surface (e.g., the hand touch surface 205). The removable films are described above in conjunction with FIG. 3. The film dispensing mechanism may include a pair of rotating cylinders, e.g., a left cylinder 410a and a right cylinder 410b. In some aspects, the cylinders 410a, 410b may be disposed at supporting pad 405 side edges, as shown in FIG. 4. In other aspects, the cylinders 410a, 410b may be disposed at supporting pad 405 top and bottom portions. Cylinder 410a, 410b lengths may be equal or equivalent to supporting pad 405 length.

In some aspects, the left cylinder 410a may include a roll of removable films. The film dispensing mechanism may automatically unroll the removable films from the left cylinder 410a, position it over the supporting pad 405 exterior surface, and may roll the used removable films on the right cylinder 410b when the removable film is used. In this case, the apparatus 400 may include pressure or proximity sensors and an apparatus 400 processor (not shown). The apparatus 400 processor may actuate left and right cylinder 410a, 410b movement (e.g., cause cylinder “rolling”) when the pressure or proximity sensors detect the user 105 using the apparatus 400 or in apparatus 400 proximity.

In further aspects, the body support apparatus 400 may include a dedicated button configured to actuate the left and right cylinder 410a, 410b movement. The dedicated button may be disposed anywhere on the supporting pad 405 exterior surface, such that the button may be easily accessible to the user 105. The user 105 may trigger or “press” the dedicated button when the user 105 uses the body support apparatus 400, and cause the left and right cylinder 410a, 410b movement and rolling of a new and fresh removable film over the supporting pad 405 exterior surface.

In some aspects, the apparatus 400 processor may rotate the left cylinder 410a and the right cylinder 410b simultaneously to move the removable film over the supporting pad 405 exterior surface. In further aspects, the apparatus 400 processor may rotate the left cylinder 410a and the right cylinder 410b at approximately the same speed.

In additional aspects, the apparatus 400 processor may rotate the cylinders 410a, 410b such that a length of unrolled removable film may be equivalent to a supporting pad 405 width, so that the removable film may completely cover the supporting pad 405 exterior surface.

In the above disclosure, reference has been made to the accompanying drawings, which form a part hereof, which illustrate specific implementations in which the present disclosure may be practiced. It is understood that other implementations may be utilized, and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a feature, structure, or characteristic is described in connection with an embodiment, one skilled in the art will recognize such feature, structure, or characteristic in connection with other embodiments whether or not explicitly described.

It should also be understood that the word “example” as used herein is intended to be non-exclusionary and non-limiting in nature. More particularly, the word “example” as used herein indicates one among several examples, and it should be understood that no undue emphasis or preference is being directed to the particular example being described.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating various embodiments and should in no way be construed so as to limit the claims.

Accordingly, it is to be understood that the above description is intended to be illustrative and not restrictive. Many embodiments and applications other than the examples provided would be apparent upon reading the above description. The scope should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

All terms used in the claims are intended to be given their ordinary meanings as understood by those knowledgeable in the technologies described herein unless an explicit indication to the contrary is made herein. In particular, use of the singular articles such as “a,” “the,” “said,” etc., should be read to recite one or more of the indicated elements unless a claim recites an explicit limitation to the contrary. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments.

That which is claimed is:

1. An apparatus comprising:
a mounting surface configured to be disposed on a structure;
a touch surface opposite to the mounting surface, wherein:
the touch surface comprises a textured hand support surface configured to provide support to a user, and
the touch surface comprises a handprint; and
a cleansing mechanism configured to clean the touch surface.

2. The apparatus of claim 1 further comprises an antibacterial or antiviral coating configured to mitigate bacterial and viral content, wherein the antibacterial or antiviral coating is disposed on the touch surface.

3. The apparatus of claim 2, wherein the antibacterial or antiviral coating comprises a copper impregnated coating.

4. The apparatus of claim 1, wherein the touch surface comprises a luminescent surface that glows in dark ambient environment.

5. The apparatus of claim 4, wherein the handprint comprises the luminescent surface that glows in the dark ambient environment.

6. The apparatus of claim 1, wherein the cleansing mechanism comprises a disinfectant light mechanism configured to disinfect the touch surface using ultraviolet light.

7. The apparatus of claim 6, wherein the cleansing mechanism further comprises at least one of: a pressure sensor and a proximity sensor.

8. The apparatus of claim 7, wherein the disinfectant light mechanism is configured to:
obtain inputs from the pressure sensor or the proximity sensor; and
disinfect the touch surface based on the inputs.

9. The apparatus of claim 1, wherein the cleansing mechanism comprises a dispenser disposed adjacent to the touch surface, wherein the dispenser comprises cleansing wipes for cleaning the touch surface.

10. The apparatus of claim 1, wherein the touch surface comprises a plurality of replaceable media disposed over the touch surface.

11. The apparatus of claim 10 further comprising a dispensing mechanism configured to change the replaceable media automatically.

12. The apparatus of claim 11, wherein the dispensing mechanism is configured to change the replaceable media responsive to a determination that a user is in proximity of the apparatus.

13. The apparatus of claim 1, wherein the structure comprises a wall.

14. The apparatus of claim 13, wherein the apparatus is mounted on the wall using adhesive.

15. The apparatus of claim 13, wherein the apparatus is mounted on the wall using fasteners.

16. An apparatus comprising:
a mounting surface configured to be disposed on a structure;
a touch surface opposite to the mounting surface, wherein:
the touch surface comprises a textured hand support surface configured to provide support to a user,
the touch surface comprises a handprint, and
the handprint comprises a luminescent surface that glows in dark ambient environment; and
a cleansing mechanism configured to clean the touch surface.

17. The apparatus of claim 16 further comprises an antibacterial or antiviral coating configured to mitigate bacterial and viral content, wherein the antibacterial or antiviral coating is disposed on the touch surface.

18. The apparatus of claim 17, wherein the antibacterial or antiviral coating comprises a copper impregnated coating.

19. The apparatus of claim 16, wherein the cleansing mechanism comprises a disinfectant light mechanism configured to disinfect the touch surface using ultraviolet light.

20. An apparatus comprising:
a mounting surface configured to be disposed on a structure;
a touch surface opposite to the mounting surface, wherein:
the touch surface comprises a textured hand support surface configured to provide support to a user, and
the touch surface comprises a handprint; and
a cleansing mechanism configured to clean the touch surface, wherein the cleansing mechanism comprises a disinfectant light mechanism configured to disinfect the touch surface using ultraviolet light.
